SEQUENCE LISTING (18 sequences)

SEQ ID NO: 1 - DNA, 20 bp, artificial sequence
  Primer dsms0003
atgaaccatggcttctcatc

SEQ ID NO: 2 - DNA, 20 bp, artificial sequence
  Primer rec0002
actgttgccggagcagacct

SEQ ID NO: 3 - DNA, 18 bp, artificial sequence
  Primer rec0003
gcattggcaactgttgcc

SEQ ID NO: 4 - DNA, 21 bp, artificial sequence
  Primer rec0004
ccggacatagcttgggtggtg

SEQ ID NO: 5 - DNA, 21 bp, artificial sequence
  Primer rec0005
gaaggtggaggatgctgtgag

SEQ ID NO: 6 - DNA, 22 bp, artificial sequence
  Primer rec0006
aggaccacctctaggcatcgga

SEQ ID NO: 7 - DNA, 20 bp, artificial sequence
  Primer rec0001
caggtctgctccggcaacag

SEQ ID NO: 8 - DNA, 20 bp, artificial sequence
  Primer dsms0004
tgattgcactaacggcatac

SEQ ID NO: 9 - DNA, 23 bp, artificial sequence
  Primer PKH1271
ccaactcaatttacctctatcca

SEQ ID NO: 10 - DNA, 22 bp, artificial sequence
  Primer PKH1274
acacccactacatacatgatca

SEQ ID NO: 11 - DNA, 24 bp, artificial sequence
  Primer flp0001
gaattcgagctcggtaccttgaag

SEQ ID NO: 12 - DNA, 23 bp, artificial sequence
  Primer flp0003
gaccatgattacgccaagcttac

SEQ ID NO: 13 - DNA, 20 bp, artificial sequence
  Primer rec0007
cattacgtgcacggccaatg

SEQ ID NO: 14 - DNA, 22 bp, artificial sequence
  Primer rec0008
ataaatgacatttgctccggct

SEQ ID NO: 15 - DNA, 24 bp, artificial sequence
  Primer rec0009
gagacatcgtgaagatagaaggca

SEQ ID NO: 16 - DNA, 20 bp, artificial sequence
  Primer rec0010
cattggccgtgcacgtaatg

SEQ ID NO: 17 - DNA, 23 bp, artificial sequence
  Primer rec0011
ccggagcaaatgtcatttatttc

SEQ ID NO: 18 - DNA, 23 bp, artificial sequence
  Primer rec0012
ttctatcttcacgatgtctcgtc

(19) United States
(12) Patent Application Publication
(10) Pub. No.: US 2019/0225988 A1
(43) Pub. Date: Jul. 25, 2019

(54) Genomic integration of DNA fragments in fungal host cells

(71) Applicant: Novozymes A/S, Bagsvaerd (DK)

(72) Inventor: Dorte M. K. Klitgaard, Bagsvaerd (DK)

(73) Assignee: Novozymes A/S, Bagsvaerd (DK)

(21) Appl. No.: 16/333,191

(22) PCT Filed: Sep. 13, 2017
(86) PCT No.: PCT/EP2017/072967

§ 371 Date: Mar. 13, 2019

(30) Foreign Application Priority Data
Sep. 15, 2016 (EP) 16189049.6

(51) Int. Cl. C12N 15/90; C12N 9/22; C12N 9/02; C12N 15/80; C12N 15/65; C07K 14/37

(52) U.S. Cl. CPC C12N 15/902; C12N 9/22; C12N 9/0036; C07K 2319/02; C12N 15/65; C07K 14/37; C07K 2319/036; C12N 15/80

(57) ABSTRACT

The instant invention relates to methods for the in vivo assembly and integration of a polynucleotide of interest at a specific chromosomal target site in a fungal host cell, said method comprising transforming the host cell with: iv. at least one polynucleotide fragment comprising an upstream flanking region and a 5′ part of the polynucleotide of interest in that order; v. at least one polynucleotide fragment comprising a 3′ part of the polynucleotide of interest and a downstream flanking region in that order; and, optionally vi. one or more additional polynucleotide fragments each comprising a 5′ part and a 3′ part of the polynucleotide of interest; wherein the 5′ part and 3′ part of each polynucleotide fragment overlap with that or those of another fragment with at least 20 bp, whereby the fragments cover the entire polynucleotide of interest, and wherein the flanking regions are of sufficient size and homology to effectuate homologous recombination with the specific target site for chromosomal integration.

### REFERENCE TO SEQUENCE LISTING

This application contains a Sequence Listing in computer readable form. The computer readable form is incorporated herein by reference.

### FIELD OF THE INVENTION

The present invention relates to methods for the in vivo assembly and integration of a polynucleotide of interest at a specific chromosomal target site in a fungal host cell, said method comprising transforming the host cell with:
i. at least one polynucleotide fragment comprising an upstream flanking region and a 5′ part of the polynucleotide of interest in that order;
ii. at least one polynucleotide fragment comprising a 3′ part of the polynucleotide of interest and a downstream flanking region in that order; and, optionally
iii. one or more additional polynucleotide fragments each comprising a 5′ part and a 3′ part of the polynucleotide of interest;

wherein the 5′ part and 3′ part of each polynucleotide fragment overlap with that or those of another fragment with at least 20 bp, whereby the fragments cover the entire polynucleotide of interest, and wherein the flanking regions are of sufficient size and homology to effectuate homologous recombination with the specific target site for chromosomal integration.

### BACKGROUND OF THE INVENTION

Methods for integrating a polynucleotide library of interest in the chromosome of a filamentous fungal host cell using a site-specific recombinase have been disclosed (WO 2016/026938).

An efficient so-called double-split marker system (DSMS) has been disclosed for site-specific and directional chromosomal integration, where the promoter and 5′ part of both the nitrite reductase (niiA) gene and nitrate reductase (niaD) gene was deleted, thereby inactivating their expression, and where an incoming construct flanked with the complementary parts of each gene is integrated by double homologous recombinations that reconstitute both genes to allow selection on minimal medium with NaNO₃ as sole nitrogen source (Nielsen M. L. et al. 2006, Efficient PCR-based gene targeting with a recyclable marker for Aspergillus nidulans, Fungal Genetics and Biology vol. 43: 54-64).

Methods for site-specific chromosomal integration of a gene library in a filamentous fungal host cell have also been disclosed that employ an autonomous replication sequence and an integration cassette comprising the gene library and a so-called split-marker or double-split marker system (DSMS) (WO2015/082535).

The general perception in the art has been that at least 500 bp are needed for efficient homologous recombination in filamentous fungal host cells, such as, in Aspergillus host cells. Consequently, it has been customary to assemble a gene library using splicing-by-overlap-extension (SOE) PCR before transforming it into a fungal host for integration.

### SUMMARY OF THE INVENTION

The invention provides methods for successfully transforming filamentous fungal Aspergillus oryzae host cells with 2-5 DNA fragments having as little as 20 bp homologous overlaps each, whereafter they recombine in vivo and are integrated site-specifically into the chromosome. Several experiments were conducted to test if the DNA fragments could be assembled in vivo having homologous overlaps of 20-1000 bp. Results showed that assembly of 2 fragments is very efficient with just a 20 bp overlap. The method was applied to lipase- and nuclease-variant generation as well as site-saturation library generation of a lipase coding sequence. A few experiments were also made with successful assembly of 3 fragments encoding a lipase. This method eliminates the need for cloning or splicing-by-overlap-extension (SOE) PCR assembly for many applications such as variant generation, library generation etc.

Accordingly, in a first aspect the invention relates to methods for the in vivo assembly and integration of a polynucleotide of interest at a specific chromosomal target site in a fungal host cell, said method comprising the steps of:
a) transforming the host cell with:
   i. at least one polynucleotide fragment comprising an upstream flanking region and a 5′ part of the polynucleotide of interest in that order;
   ii. at least one polynucleotide fragment comprising a 3′ part of the polynucleotide of interest and a downstream flanking region in that order; and, optionally
   iii. one or more additional polynucleotide fragment each comprising a 5′ part and a 3′ part of the polynucleotide of interest;
   wherein the 5′ part and 3′ part of each polynucleotide fragment overlap with that or those of another fragment with at least 20 bp, whereby the fragments cover the entire polynucleotide of interest, and wherein the flanking regions are of sufficient size and homology to the specific chromosomal target site to effectuate homologous recombinations with the specific target site;
b) cultivating the transformed host cell under conditions conducive for in vivo homologous recombinations to take place, wherein the overlapping PCR fragments recombine to form the entire polynucleotide of interest and the flanking regions recombine with the chromosome to integrate the polynucleotide of interest at the specific chromosomal target site; and
c) selecting a transformed host cell, wherein the polynucleotide of interest is integrated into the specific chromosomal target site.

### BRIEF DESCRIPTION OF DRAWINGS

FIG. 1 shows an illustration of the transformation substrates (Fragment A-G) for in vivo assembly of two fragments with overlaps of 20-1000 bp, as described in Example 1 and Example 2.

FIG. 2 shows an illustration of the transformation substrates (Fragment A-I) for in vivo assembly of two to five fragments with overlaps of exactly 20 bp, as described in Example 3.

FIG. 3 shows an illustration of the transformation reactions (Reaction I-V) for in vivo assembly of two to five fragments with overlaps of exactly 20 bp, as described in Example 3.

### DEFINITIONS

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

### DETAILED DESCRIPTION OF THE INVENTION

In a first aspect, the invention relates to methods for the in vivo assembly and integration of a polynucleotide of interest at a specific chromosomal target site in a fungal host cell, said method comprising the steps of:
a) transforming the host cell with:
   i. at least one polynucleotide fragment comprising an upstream flanking region and a 5′ part of the polynucleotide of interest in that order;
   ii. at least one polynucleotide fragment comprising a 3′ part of the polynucleotide of interest and a downstream flanking region in that order; and, optionally
   iii. one or more additional polynucleotide fragment each comprising a 5′ part and a 3′ part of the polynucleotide of interest;
   wherein the 5′ part and 3′ part of each polynucleotide fragment overlap with that or those of another fragment with at least 20 bp, whereby the fragments cover the entire polynucleotide of interest, and wherein the flanking regions are of sufficient size and homology to the specific chromosomal target site to effectuate homologous recombinations with the specific target site;
b) cultivating the transformed host cell under conditions conducive for in vivo homologous recombinations to take place, wherein the overlapping PCR fragments recombine to form the entire polynucleotide of interest and the flanking regions recombine with the chromosome to integrate the polynucleotide of interest at the specific chromosomal target site; and
c) selecting a transformed host cell, wherein the polynucleotide of interest is integrated into the specific chromosomal target site.

### Host Cells

The present invention also relates to recombinant host cells, comprising a gene of interest encoding a polypeptide or enzyme of interest. The gene of interest may be operably linked to one or more control sequences that direct the production of the polypeptide. A construct or vector comprising a polynucleotide is introduced into the host cell so that the construct or vector is maintained as a chromosomal integrant. The term “host cell” encompasses any progeny of a parent cell that is not identical to the parent cell due to mutations that occur during replication.

The choice of a host cell will to a large extent depend upon the gene encoding the polypeptide and its source.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulaturn, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787.

For the purposes of the instant invention, it is advantageous to employ a fungal host cell which is non-homologous end joining repair deficient; preferably the host cell comprises an inactivating mutation or a deletion in a gene required for non-homologous end-joining repair; more preferably the host cell comprises an inactivating mutation in kusA, IigD, ku70, ku80, mre11, rad50 and/or xrs2, or an evolutionary homologue thereof; most preferably kusA, IigD, ku70, ku80, mre11, rad50 and/or xrs2, or an evolutionary homologue thereof, is/are deleted from the fungal host cell.

In a preferred embodiment of the first aspect, the specific chromosomal target site of the fungal host cell and the upstream and downstream flanking regions together comprise a double split-selection marker system (DSMS), thereby allowing the selection of a transformed host cell, wherein the polynucleotide of interest is integrated into the specific chromosomal target site in a specific orientation; preferably the double split-selection marker system is based on split nitrite reductase (nhA) and nitrate reductase (niaD) genes or evolutionary homologues thereof in the host cell, thereby allowing the selection of a successfully transformed host cell on minimal media supplemented with NaNO₃, wherein the polynucleotide of interest is integrated into the specific chromosomal target site in a specific orientation, while reconstituting the nitrite reductase (nhA) and nitrate reductase in the process.

In another preferred embodiment, the host cell is transformed in step (a) with at most 100 additional polynucleotide fragments; preferably at most 90; 80; 70; 60; 50; 40; 30 or 20 additional polynucleotide fragments; more preferably at most 10 additional polynucleotide fragments; even more preferably at most 5 additional polynucleotide fragments; most preferably at most 1 additional polynucleotide fragment.

### Polynucleotides

The present invention also relates to polynucleotides comprising a gene encoding an enzyme of interest; preferably the polynucleotide of interest comprises a gene encoding an enzyme comprising a secretion signal peptide; more preferably the polynucleotide of interest comprises a gene encoding a hydrolase, isomerase, ligase, lyase, oxidoreductase, or transferase; most preferably the polynucleotide of interest comprises a gene encoding an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

In a preferred embodiment, one or more of the polynucleotide fragments transformed into the host cell comprises at least one mutation in the gene encoding an enzyme of interest, whereby, when the overlapping PCR fragments recombine to form the entire polynucleotide of interest, the at least one mutation will be comprised in the gene encoding an enzyme of interest.

In another preferred embodiment, the polynucleotide of interest comprises a selectable marker gene.

Preferably, the 5′ part and 3′ part of each polynucleotide fragment of the first aspect of the invention overlap with that or those of another fragment with at most 1,000 bp; preferably at most 900 bp; at most 800 bp; at most 700 bp; at most 600 bp; at most 500 bp; at most 400 bp; at most 300 bp; at most 200 bp; at most 100 bp; at most 90 bp; at most 80 bp; at most 70 bp; at most 60 bp; at most 50 bp; at most 40 bp; and most preferably the 5′ part and 3′ part of each polynucleotide fragment overlap with that or those of another fragment with at most 30 bp.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

The control sequence may also be an mRNA stabilizer region downstream of a promoter and upstream of the coding sequence of a gene which increases expression of the gene. The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph−tk dual selectable marker system.

The vector preferably contains an element(s) that permits integration of the vector into the host cell's genome or autonomous replication of the vector in the cell independent of the genome. For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### EXAMPLES

### Materials and Methods

All DNA for transformation was amplified using a proof-reading polymerase, Phusion (Thermo Fisher Scientific), according to the manufacturer's instructions. DNA was purified using the QIAquick kit (QIAGEN®), according to the manufacturer's instructions.

DNA for sequencing was amplified using a proof-reading polymerase, KAPA HotStart ReadyMix (KAPA Biosystems), according to the manufacturer's instructions.

Aspergillus oryzae COLS1300 (amyA, amyB, amyC, alpA, nprA, kusA, niaD, niiA, amdS+) was created from A. oryzae PFJ0220 (EP2147107B1) by deleting the promoter and 5′ part of both the nitrite reductase (niiA) gene and nitrate reductase (niaD) gene, thereby inactivating their expression (Nielsen M. L. et al. 2006, Efficient PCR-based gene targeting with a recyclable marker for Aspergillus nidulans, Fungal Genetics and Biology vol. 43: 54-64).

Aspergillus oryzae strain DAU716 (ligDΔ) is described in WO 2016/026938, Example 5.

Transformation of fungal species can be achieved using the general methods for yeast transformation. Preferably, Aspergillus host strains are inoculated into 100 ml YPG medium supplemented with 10 mM uridine and incubated for 16 hrs at 32° C. at 80 rpm. Pellets were collected and washed with 0.6 M KCl, and resuspended in 20 ml 0.6 M KCl containing a commercial β-glucanase product (GLUCAN EX™, Novozymes A/S, Bagsvaerd, Denmark) at a final concentration of 20 mg per ml. The suspension was incubated at 32° C. with shaking (80 rpm) until protoplasts were formed, and then washed twice with STC buffer. The protoplasts were counted, resuspended and adjusted in an 8:2:0.1 solution of STC:STPC:DMSO to a final concentration of 2.5×10⁷ protoplasts/ml. Approximately 4 μg of plasmid DNA was added to 100 μl of the protoplast suspension, mixed gently, and incubated on ice for 30 minutes. One ml of SPTC was added and the protoplast suspension was incubated for 20 minutes at 37° C. After the addition of 10 ml of 50° C. Cove or Cove-N top agarose, the reaction was poured onto Cove or Cove-N (if) agar plates and the plates were incubated at 32° C. for 5 days.

### Example 1. Transforming 2 Fragments into A. oryzae by Double-Split Marker

The purpose of this experiment was to show that in vivo assembly of two fragments is possible without direct selection for the assembly event, unlike the previously described double split marker system (DSMS), where selection is directly applied for assembly of the selection marker (Nielsen M. L. et al. 2006, Efficient PCR-based gene targeting with a recyclable marker for Aspergillus nidulans, Fungal Genetics and Biology vol. 43: 54-64). However, indirect selection for in vivo assembly was applied, as the DSMS system selects for integration in both the 5′ end and the 3′ end of the integration site.

Furthermore, the purpose was to show that in vivo assembly of two fragments is possible when having as little as 20 bp homology overlap between the two fragments. In this example, the homology overlap is identical to the wild-type gene, but the homology overlap could contain any nucleotide sequence and could, e.g., be used for introduction of specific mutations.

Preparation of Fragments with 20, 30, 50, 100, and 1000 bp Overlap:

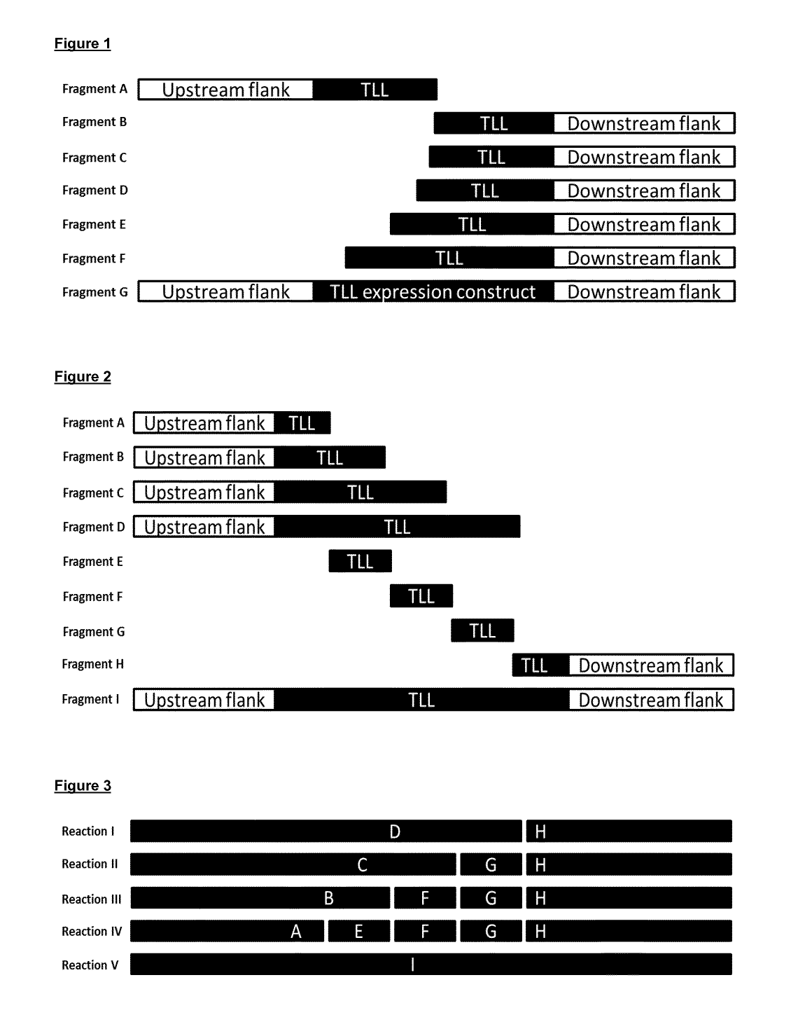

Two PCR fragments with different length of overlap for integration by homologous recombination (HR) were produced as described below and illustrated in FIG. 1. The system was tested by expressing the Thermomyces lanuginosus lipase integrated via the double split marker system (DSMS; vide supra). The PCR fragments were designed to have overlaps of 20 bp, 30 bp, 50 bp, 100 bp, and 1000 bp, respectively. The fragments were amplified by PCR from pBGMH0021 (WO2015/082535) using the primers pairs of table 1:

TABLE 1

| Fragment | Forward primer 5′→3′ | Reverse primer 5′→3′ |
|---|---|---|
| A: Universal upstream fragment for | dsms0003: | rec0001 |
| all constructs | atgaaccatggcttctcatc | caggtctgctccggcaacag |
| | (SEQ ID NO: 1) | (SEQ ID NO: 7) |
| B: Downstream fragment with 20 bp | rec0002: | dsms0004: |
| overlap to upstream fragment | actgttgccggagcagacct | tgattgcactaacggcatac |
| | (SEQ ID NO: 2) | (SEQ ID NO: 8) |
| C: Downstream fragment with 30 bp | rec0003: | dsms0004: |
| overlap to upstream fragment | gcattggcaactgttgcc | tgattgcactaacggcatac |
| | (SEQ ID NO: 3) | (SEQ ID NO: 8) |
| D: Downstream fragment with 50 bp | rec0004: | dsms0004: |
| overlap to upstream fragment | ccggacatagcttgggtggtg | tgattgcactaacggcatac |
| | (SEQ ID NO: 4) | (SEQ ID NO: 8) |
| E: Downstream fragment with 100 | rec0005: | dsms0004: |
| bp overlap to upstream fragment | gaaggtggaggatgctgtgag | tgattgcactaacggcatac |
| | (SEQ ID NO: 5) | (SEQ ID NO: 8) |
| F: Downstream fragment with 1000 | rec0006: | dsms0004: |
| bp overlap to upstream fragment | aggaccacctctaggcatcgga | tgattgcactaacggcatac |
| | (SEQ ID NO: 6) | (SEQ ID NO: 8) |
| G: Complete fragment as control, | dsms0003: | dsms0004: |
| containing both upstream and | atgaaccatggcttctcatc | tgattgcactaacggcatac |
| downstream sequence | (SEQ ID NO: 1) | (SEQ ID NO: 8) |

The PCR reactions were programmed for 1 cycle at 95° C. for 5 minutes; 35 cycles each at 98° C. for 30 seconds, 58° C. for 30 seconds, and 72° C. for 4 minutes; and a final elongation at 72° C. for 5 minutes.
Transformation of COLS1300 (kusAΔ):

The fragments were transformed pairwise into COLS1300 as shown below, and as illustrated in FIG. 1. Transformants were plated on to plates with minimal media supplemented with NaN0₃. Only spores, where the integration cassette had successfully recombined into the chromosome of the host cell and reconstituted the niiA and niaD sites in the process, could germinate and survive. The number of transformants was counted after 3 days incubation at 30° C.

| | Overlap | Transformants/μg |
|---|---|---|
| Fragments | length (bp) | DNA |
| A + B | 20 | 11 |
| A + C | 30 | 17 |
| A + D | 50 | 60 |
| A + E | 100 | 42 |
| A + F | 1000 | 23 |
| G (positive control) | Single fragment | 30 |
| No DNA (negative | — | 0 |
| control) | | |

### Sequencing of Transformants:

The Thermomyces lanuginosus lipase was PCR amplified from genomic DNA of 8 transformants from each reaction using the primers:

PKH1271:
(SEQ ID NO: 9)
5′ ccaactcaatttacctctatcca
PKH1274:
(SEQ ID NO: 10)
5′ acacccactacatacatgatca

The PCR reactions were programmed for 1 cycle at 95° C. for 3 minutes; 34 cycles each at 98° C. for 20 seconds, 68° C. for 1 minute, and 72° C. for 1 minute; and a final elongation at 72° C. for 4 minutes.

### Results

The Aspergillus oryzae COLS1300 strain is kusAΔ (deficient in non-homologous end-joining repair (NHEJ). In this strain it was possible to assemble two fragments in vivo, without applying direct selection for the assembly event. Furthermore, it was possible to assemble two fragments in vivo, containing as little as 20 bp homology overlap for HR. Sequencing verified that all tested transformants had integrated both fragments, and no mutations had been introduced during the HR events.

### Example 2. Transformation with Two Fragments in A. oryzae by FLP/FRT System

The purpose of this experiment was to show that in vivo assembly of two transformed PCR fragments is possible without direct selection and without indirect selection for integration of both fragments. The FLP/FRT system selects for integration in the 3′ end of the integration site only, and therefore there is no selection for assembly of the two fragments.

Preparation of Fragments with 20, 30, 50, 100, and 1000 bp Overlap:

Fragments for integrating two fragments with different length of overlap for HR were produced in the following manner. The system was tested by expressing the Thermomyces lanuginosus lipase via the site-specific recombinase FLP/FRT system (WO 2012/160093). The fragments were designed to have overlaps of 20 bp, 30 bp, 50 bp, 100 bp, and 1000 bp, respectively. The fragments were amplified by PCR from plasmid pDAU724 (WO 2016/026938; FIG. 7; SEQ ID NO:30 therein) using the primer pairs of table 2:

TABLE 2

| Fragment | Forward primer 5′→3′ | Reverse primer 5′→3′ |
|---|---|---|
| A: Universal upstream fragment | flp0001: | rec0001: |
| for all constructs | gaattcgagctcggtaccttgaag | caggtctgctccggcaacag |
| | (SEQ ID NO: 11) | (SEQ ID NO: 7) |
| B: Downstream fragment with 20 | rec0002: | flp0003: |
| bp overlap to upstream fragment | actgttgccggagcagacct | gaccatgattacgccaagcttac |
| | (SEQ ID NO: 2) | (SEQ ID NO: 12) |
| C: Downstream fragment with 30 | rec0003: | flp0003: |
| bp overlap to upstream fragment | gcattggcaactgttgcc | gaccatgattacgccaagcttac |
| | (SEQ ID NO: 3) | (SEQ ID NO: 12) |
| D: Downstream fragment with 50 | rec0004: | flp0003: |
| bp overlap to upstream fragment | ccggacatagcttgggtggtg | gaccatgattacgccaagcttac |
| | (SEQ ID NO: 4) | (SEQ ID NO: 12) |
| E: Downstream fragment with | rec0005: | flp0003: |
| 100 bp overlap to upstream | gaaggtggaggatgctgtgag | gaccatgattacgccaagcttac |
| fragment | (SEQ ID NO: 5) | (SEQ ID NO: 12) |
| F: Downstream fragment with | rec0006: | flp0003: |
| 1000 bp overlap to upstream | aggaccacctctaggcatcgga | gaccatgattacgccaagcttac |
| fragment | (SEQ ID NO: 6) | (SEQ ID NO: 12) |
| G: Complete fragment as control, | flp0001: | flp0003: |
| containing both upstream and | gaattcgagctcggtaccttgaag | gaccatgattacgccaagcttac |
| downstream sequence | (SEQ ID NO: 11) | (SEQ ID NO: 12) |

The PCR reactions were programmed for 1 cycle at 95° C. for 5 minutes; 35 cycles each at 98° C. for 30 seconds, 60° C. for 30 seconds, and 72° C. for 3 minutes; and a final elongation at 72° C. for 5 minutes.

Transformation of A. oryzae Strain DAU716 (ligDΔ):

The fragments were transformed pairwise into DAU716 as shown in table 3 and as illustrated in FIG. 1, and the transformants were counted after 3 days incubation at 30° C.

TABLE 3

| | Overlap | Transformants/μg |
|---|---|---|
| Fragments | length (bp) | DNA |
| A + B | 20 | 8 |
| A + C | 30 | 24 |
| A + D | 50 | 21 |
| A + E | 100 | 18 |
| A + F | 1000 | 4 |
| G (linear positive control) | Single fragment | 0 |
| pDAU724 (circular | Single circular | 35 |
| positive control) | plasmid | |
| No DNA (negative control) | — | 0 |

### Sequencing of Transformants:

The Thermomyces lanuginosus lipase-encoding gene was PCR amplified from genomic DNA of 8 transformants from each reaction using the primers:

PKH1271:
(SEQ ID NO: 9)
5′ ccaactcaatttacctctatcca
PKH1274:
(SEQ ID NO: 10)
5′ acacccactacatacatgatca

The PCR reactions were programmed for 1 cycle at 95° C. for 3 minutes; 34 cycles each at 98° C. for 20 seconds, 68° C. for 1 minute, and 72° C. for 1 minute; and a final elongation at 72° C. for 4 minutes.

### Results

Aspergillus oryzae DAU716 is ligDΔ (deficient in NHEJ). We did not apply direct or indirect selection for assembly and integration of two fragments, yet it was possible to assemble two fragments in vivo, where the two fragments contained as little as 20 bp homology overlap for homologous recombination in vivo.

Sequencing verified that all transformants had integrated both fragments, and no mutations had been introduced during the recombinations. Thus, correct assembly of two fragments with short homologous overlaps is not restricted to using a specific integration system.

### Example 3. Transformation with 2-5 Fragments in A. oryzae by the DSMS System

The purpose of this experiment was to show that in vivo assembly of up to five fragments is possible without direct selection for integration of all fragments, when having homologous overlaps of only 20 bp.

Preparation of Fragments with 20 bp Overlap:

2-5 fragments for integration with 20 bp overlaps were produced in the following manner, and as illustrated in FIG. 2. The system was tested by expressing the Thermomyces lanuginosus lipase via the double split marker system (DSMS). The fragments were designed to integrate 2-5 fragments, and they all had overlaps of exactly 20 bp. The fragments were amplified by PCR from pBGMH0021, using the primer pairs of table 4:

TABLE 4

| Fragment | Forward primer 5′→3′ | Reverse primer 5′→3′ |
|---|---|---|
| A: Upstream fragment | dsms0003: | rec0010: |
| 0-200 bp | atgaaccatggcttctcatc | cattggccgtgcacgtaatg |
| | (SEQ ID NO: 1) | (SEQ ID NO: 16) |
| B: Upstream fragment | dsms0003: | rec0011: |
| 0-400 bp | atgaaccatggcttctcatc | ccggagcaaatgtcatttatttc |
| | (SEQ ID NO: 1) | (SEQ ID NO: 17) |
| C: Upstream fragment | dsms0003: | rec0001: |
| 0-600 bp | atgaaccatggcttctcatc | caggtctgctccggcaacag |
| | (SEQ ID NO: 1) | (SEQ ID NO: 7) |
| D: Upstream fragment | dsms0003: | rec0012: |
| 0-800 bp | atgaaccatggcttctcatc | ttctatcttcacgatgtctcgtc |
| | (SEQ ID NO: 1) | (SEQ ID NO: 18) |
| E: Middle fragment, | rec0007: | rec0011: |
| 200-400 bp | cattacgtgcacggccaatg | ccggagcaaatgtcatttatttc |
| | (SEQ ID NO: 13) | (SEQ ID NO: 17) |
| F: Middle fragment, | rec0008: | rec0001: |
| 400-600 bp | ataaatgacatttgctccggct | caggtctgctccggcaacag |
| | (SEQ ID NO: 14) | (SEQ ID NO: 7) |
| G: Middle fragment, | rec0002: | rec0012: |
| 600-800 bp | actgttgccggagcagacct | ttctatcttcacgatgtctcgtc |
| | (SEQ ID NO: 2) | (SEQ ID NO: 18) |
| H: Universal | rec0009: | dsms0004: |
| downstream fragment | gagacatcgtgaagatagaaggca | tgattgcactaacggcatac |
| | (SEQ ID NO: 15) | (SEQ ID NO: 8) |
| I: Complete fragment | dsms0003: | dsms0004: |
| as control, containing | atgaaccatggcttctcatc | tgattgcactaacggcatac |
| both upstream and | (SEQ ID NO: 1) | (SEQ ID NO: 8) |
| downstream sequence | | |

The PCR reactions were programmed for 1 cycle at 95° C. for 5 minutes; 35 cycles each at 98° C. for 30 seconds, 60° C. for 30 seconds, and 72° C. for 3 minutes; and a final elongation at 72° C. for 5 minutes.

Transformation of A. oryzae COLS1300 (kusAΔ):

The fragments were transformed pairwise into COLS1300 as shown in table 5 and as illustrated in FIG. 3; the transformants were counted after 3 days incubation at 30° C.

TABLE 5

| | | Overlap length | Transformants |
|---|---|---|---|
| Reaction | Fragments | (bp) | per μg DNA |
| I | D + H | 20 | 25 |
| II | C + G + H | 20 | 6 |
| III | B + F + G + H | 20 | 3 |
| IV | A + E + F + G + H | 20 | 0.3 |
| V | I (positive control) | 20 | 2 |
| | No DNA (negative | — | 0 |
| | control) | | |

### Sequencing of Transformants:

The Thermomyces lanuginosus lipase was PCR amplified from genomic DNA of 8 transformants from each reaction using the primers:

PKH1271:
(SEQ ID NO: 9)
5′ ccaactcaatttacctctatcca
PKH1274:
(SEQ ID NO: 10)
5′ acacccactacatacatgatca

The PCR reactions were programmed for 1 cycle at 95° C. for 3 minutes; 34 cycles each at 98° C. for 20 seconds, 68° C. for 1 minute, and 72° C. for 1 minute; and a final elongation at 72° C. for 4 minutes.

### Results

Aspergillus oryzae COLS1300 is kusAΔ (deficient of NHEJ). In this strain it was possible to assemble up to 5 transformed PCR fragments in vivo, each containing as little as 20 bp overlap with another fragment for homologous recombination. Sequencing verified that all tested transformants had integrated all fragments, and no mutations had been introduced during the HR event.

It was observed that transformation efficiency decreased with increasing number of fragments; however, it may be possible to assemble a higher number of fragments if the general transformation efficiency of the host is increased.

1. A method for the in vivo assembly and integration of a polynucleotide of interest at a specific chromosomal target site in a fungal host cell, said method comprising the steps of:
a) transforming the host cell with:
i. at least one polynucleotide fragment comprising an upstream flanking region and a 5′ part of the polynucleotide of interest in that order;
ii. at least one polynucleotide fragment comprising a 3′ part of the polynucleotide of interest and a downstream flanking region in that order; and, optionally
iii. one or more additional polynucleotide fragment each comprising a 5′ part and a 3′ part of the polynucleotide of interest;
wherein the 5′ part and 3′ part of each polynucleotide fragment overlap with that or those of another fragment with at least 20 bp, whereby the fragments cover the entire polynucleotide of interest, and wherein the flanking regions are of sufficient size and homology to the specific chromosomal target site to effectuate homologous recombinations with the specific target site;
b) cultivating the transformed host cell under conditions conducive for in vivo homologous recombinations to take place, wherein the overlapping PCR fragments recombine to form the entire polynucleotide of interest and the flanking regions recombine with the chromosome to integrate the polynucleotide of interest at the specific chromosomal target site; and
c) selecting a transformed host cell, wherein the polynucleotide of interest is integrated into the specific chromosomal target site.

2. The method of claim 1, wherein the fungal host cell is a filamentous fungal host cell, preferably the filamentous fungal host cell is of the genus Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma; even more preferably the filamentous fungal host cell is an Aspergillus awamori, Aspergillus foetidus, Aspergillus fumigatus, Aspergillus japonicus, Aspergillus nidulans, Aspergillus niger or Aspergillus oryzae host cell.

3. The method of claim 1, wherein the fungal host cell is non-homologous end joining repair deficient; preferably the host cell comprises an inactivating mutation or a deletion in a gene required for non-homologous end-joining repair; more preferably the host cell comprises an inactivating mutation in kusA, ligD, ku70, ku80, mre11, rad50 and/or xrs2, or an evolutionary homologue thereof; most preferably kusA, ligD, ku70, ku80, mre11, rad50 and/or xrs2, or an evolutionary homologue thereof, is/are deleted from the fungal host cell.

4. The method of claim 1, wherein the 5′ part and 3′ part of each polynucleotide fragment overlap with that or those of another fragment with at most 1,000 bp; preferably at most 900 bp; at most 800 bp; at most 700 bp; at most 600 bp; at most 500 bp; at most 400 bp; at most 300 bp; at most 200 bp; at most 100 bp; at most 90 bp; at most 80 bp; at most 70 bp; at most 60 bp; at most 50 bp; at most 40 bp; and most preferably the 5′ part and 3′ part of each polynucleotide fragment overlap with that or those of another fragment with at most 30 bp.

5. The method of claim 1, wherein the polynucleotide of interest comprises a gene encoding an enzyme of interest; preferably the polynucleotide of interest comprises a gene encoding an enzyme comprising a secretion signal peptide; more preferably the polynucleotide of interest comprises a gene encoding a hydrolase, isomerase, ligase, lyase, oxidoreductase, or transferase; most preferably the polynucleotide of interest comprises a gene encoding an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

6. The method of claim 5, wherein one or more of the polynucleotide fragments transformed into the host cell comprises at least one mutation in the gene encoding an enzyme of interest, whereby, when the overlapping PCR fragments recombine to form the entire polynucleotide of interest, the at least one mutation will be comprised in the gene encoding an enzyme of interest.

7. The method of claim 1, wherein the polynucleotide of interest comprises a selectable marker gene.

8. The method of claim 1, wherein the specific chromosomal target site of the fungal host cell and the upstream and downstream flanking regions together comprise a double split-selection marker system (DSMS), thereby allowing the selection of a transformed host cell, wherein the polynucleotide of interest is integrated into the specific chromosomal target site in a specific orientation; preferably the double split-selection marker system is based on split nitrite reductase (niiA) and nitrate reductase (niaD) genes or evolutionary homologues thereof in the host cell, thereby allowing the selection of a successfully transformed host cell on minimal media supplemented with NaN0₃, wherein the polynucleotide of interest is integrated into the specific chromosomal target site in a specific orientation, while reconstituting the nitrite reductase (niiA) and nitrate reductase in the process.

9. The method of claim 1, wherein the host cell is transformed in step (a) with at most 100 additional polynucleotide fragments; preferably at most 90; 80; 70; 60; 50; 40; 30 or 20 additional polynucleotide fragments; more preferably at most 10 additional polynucleotide fragments; even more preferably at most 5 additional polynucleotide fragments; most preferably at most 1 additional polynucleotide fragment.
